(19) United States
(12) Patent Application Publication
(10) Pub. No.: US 2009/0227859 A1
(43) Pub. Date: Sep. 10, 2009

(54) SYSTEMS AND METHODS FOR DETERMINING A CARDIOVASCULAR PARAMETER USING TEMPERATURE SENSITIVE MAGNETIC RESONANCE IMAGING

(76) Inventors: John Pile-Spellman, Pelham, NY (US); Erwin Lin, Whitestone, NY (US)

Correspondence Address: KENYON & KENYON LLP, ONE BROADWAY, NEW YORK, NY, 10004, US

(21) Appl. No.: 12/161,507

(22) PCT Filed: Jan. 22, 2007
(86) PCT No.: PCT/US07/01795

§ 371 Date: Nov. 17, 2008

Related U.S. Application Data
(60) Provisional application No. 60/761,755, filed on Jan. 25, 2006.

(51) Int. Cl. A61B 5/055

(52) U.S. Cl. 600/419

(57) ABSTRACT

A method for determining a cardiovascular parameter in a portion of a body of a patient utilizing temperature sensitive MRI measurements. The method includes obtaining magnetic resonance information from a portion of a body of a patient and determining a magnetic resonance parameter using the magnetic resonance information. The method further includes using the magnetic resonance parameter to determine a temperature differential in the portion of the body and determining a cardiovascular parameter using the temperature differential.

### CROSS-REFERENCE TO RELATED APPLICATION

The present application claims the benefit of and priority to International Patent Application No. PCT/U.S.07/01795, filed 22 Jan. 2007, which claims the benefit of and priority to U.S. Provisional Patent Application No. 60/761,755, filed 25 Jan. 2006, both of which are expressly incorporated herein in their entireties by reference thereto.

The present application is related to co-pending applications “Systems and Methods for Imaging a Blood Vessel Using Temperature Sensitive Magnetic Resonance Imaging,” filed herewith and “Systems and Methods for Determining Metabolic Rate Using Temperature Sensitive Magnetic Resonance Imaging,” filed herewith. Both applications are incorporated by reference herein.

### FIELD OF THE INVENTION

The present invention relates to systems and methods for determining a cardiovascular parameter based on a temperature differential determined from information obtained by magnetic resonance imaging.

### BACKGROUND

Tissue perfusion is a measure of the delivery of blood to a part of the body. While perfusion to an organ can be viewed on a global level, such as perfusion to an entire organ, perfusion can also be viewed on a local level, such as perfusion to a small region. Many disease processes cause perfusion abnormalities at a global or local level and measurement of absolute and relative values of tissue perfusion have been used to diagnose disease and to assess the stage, degree and reversibility of disease. Non-invasive methods to measure tissue perfusion include magnetic resonance imaging (“MRI”), computerized tomography (“CT”), ultrasound (“US”) and nuclear medicine

These non-invasive methods rely primarily on dilution of an indicator or tracer introduced into a blood vessel. Specifically, a substance is introduced into the cardiovascular system and the concentration of the indicator in a voxel or a larger region is measured to calculate parameters that reflect relative or absolute measures of tissue perfusion. The concentration of an indicator within a voxel is determined by the quantity of indicator delivered to the voxel, the transit time of the indicator through the voxel and the volume of distribution of the indicator within the voxel.

Indicators may be diffusible or non-diffusible based on their physical properties as well as the physical characteristics of the vessels and tissue being perfused. Non-diffusible indicators, such as gadolinium contrast agents used in the brain, remain confined to blood vessels and their concentration is therefore dependent on the volume of blood vessels (i.e., the “blood volume”) within the voxel. Diffusible indicators, such as gadolinium contrast agents used outside of the central nervous system or labeled protons using arterial spin labeling, can freely diffuse into the voxel interstitium and their concentration is therefore determined by the sum of the blood volume and the interstitial volume of the voxel.

Whether using a currently available non-diffusible or diffusible indicator, a variety of assumptions and estimations may have to be made when using MRI to measure tissue perfusion. Specifically, assumptions may have to be made to calculate tissue concentration from MR signal or phase change measurements. For example, when using gadolinium contrast agents in the brain, assuming T1 effects can be ignored results in a linear relationship between local tissue concentration of gadolinium and changes in T2 relaxation. Assumptions and estimations are a potential source of error when the calculated tissue concentrations are then used to calculate cardiovascular parameters such as flow, volume of distribution and mean transit time. When using arterial spin labeling, calculations used to obtain tissue concentration of labeled spins based on MR signal measurements require complex alterations of the Bloch equations. Furthermore, unless the arterial input function is known, such as by using an intra-arterial injection of indicator through a catheter, or measured in a major artery supplying the tissue of interest, only relative values of the flow to volume ratio may be calculated, regardless of the technique utilized.

When using gadolinium-based techniques, only a single dose of gadolinium contrast agent can typically be administered at any one time due to safety concerns. In addition, gadolinium contrast agents are expensive.

A need therefore exists for a MI method and system for measuring perfusion using a diffusible indicator that has more ideal properties and allows simpler and more accurate calculations.

### SUMMARY OF THE INVENTION

Systems and methods for determining a cardiovascular parameter using temperature sensitive magnetic resonance imaging are provided. In an embodiment, the present invention provides a method for determining a cardiovascular parameter of a portion of a body of a patient. The method comprises introducing a fluid into a blood vessel of the patient and obtaining magnetic resonance information from the portion of the body. The method further comprises determining a magnetic resonance parameter from the portion of the body using the magnetic resonance information and determining a temperature differential in the portion of the body using the magnetic resonance parameter. The method further comprises determining the cardiovascular parameter using the temperature differential.

In an embodiment, the present invention provides a machine-readable medium having stored thereon a plurality of executable instructions, which, when performed by a processor, performs obtaining magnetic resonance information from a portion of a body of a patient after introduction of fluid into a blood vessel of the patient and determining a magnetic resonance parameter from the portion of the body using the magnetic resonance information. The plurality of executable instructions further performs determining a temperature differential in the portion of the body using the magnetic resonance parameter and determining a cardiovascular parameter using the temperature differential.

### BRIEF DESCRIPTION OF THE DRAWINGS

The present invention will become more fully understood from the detailed description given herein below and the accompanying drawings which are given by way of illustration only and wherein:

FIG. 1 is a flow diagram that illustrates an embodiment of a method of measuring a cardiovascular parameter using temperature sensitive MRI.

FIG. 2 depicts an embodiment of a system for controlling the temperature of a fluid that is introduced into a patient.

FIG. 3 is a block diagram that depicts an embodiment of a user computing device

FIG. 4 is a block diagram that depicts an embodiment of a network architecture.

FIG. 5 is a graph of temperature changes in a capillary phantom as a function of time, calculated according to an embodiment of the invention, using sequential dynamic phase images following an injection of a cold saline bolus. Temperature change with respect to baseline (room temperature) is shown on the vertical axis in units of degrees Centigrade Time, represented by image number (where the time between images is a fixed constant) is shown on the horizontal axis increasing from left to right.

FIG. 6 is a graph showing the measured temperature as a function of time at a thermometer 1 (A) and a thermometer 2 (B) that corresponds to the cold saline bolus of FIG. 5. The baseline temperature is slightly greater than 21° C.

FIG. 7 is a graph of calculated temperature changes in a capillary phantom as determined by sequential dynamic phase images as a function of time following an injection of a room temperature saline bolus. Temperature change with respect to baseline (room temperature) is shown on the vertical axis in units of degrees Centigrade. Time, represented by image number (where the time between images is a fixed constant) is shown on the horizontal axis increasing from left to right.

FIG. 8 is a graph showing the measured temperature as a function of time at a thermometer 1 (A) and a thermometer 2 (B) that corresponds to the room temperature bolus of FIG. 7. The baseline temperature is slightly greater than 21° C.

### DETAILED DESCRIPTION OF THE INVENTION

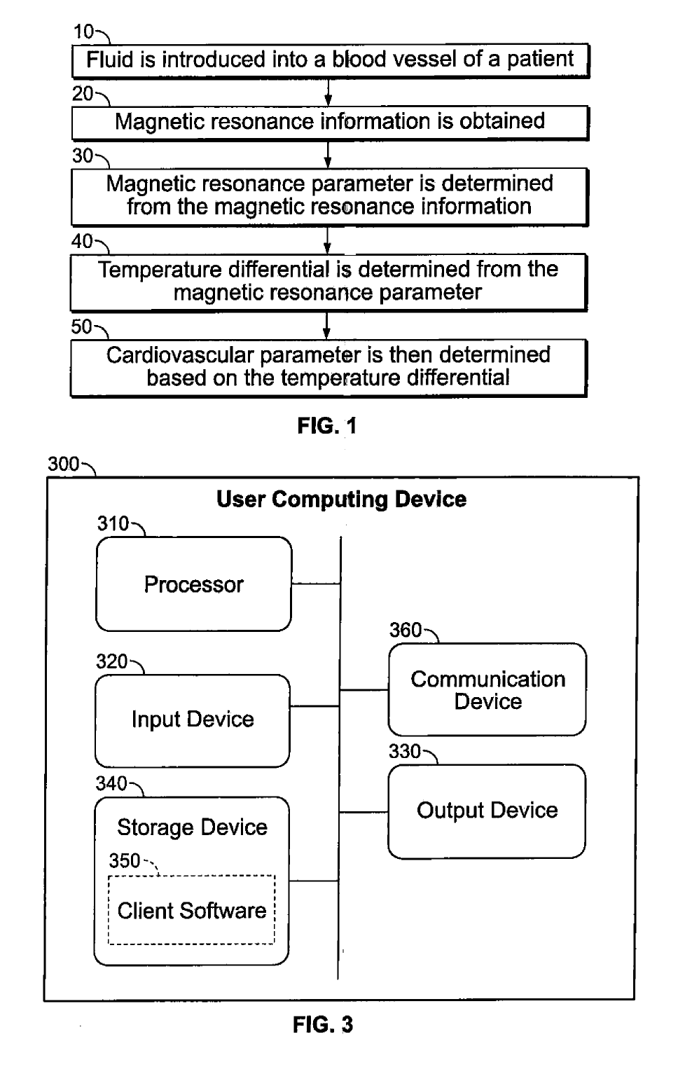

In an embodiment, the present invention provides a method for determining a cardiovascular parameter in a portion of a body of a patient based on a temperature differential of the portion of the body determined from information obtained by MRI. Specifically, referring to FIG. 1, a method for determining a cardiovascular parameter comprises introducing a fluid into a blood vessel of a patient (10) and then obtaining magnetic resonance information from a portion of the body of the patient (20). A magnetic resonance parameter is determined using the magnetic resonance information (30) and a temperature differential in the portion of the body is determined using the magnetic resonance parameter (40). Based on the temperature differential, a cardiovascular parameter is determined (50).

A cardiovascular parameter that is determined in a portion of the body can be any cardiovascular parameter (qualitative and/or quantitative) associated with tissue perfusion. Non-limiting examples of cardiovascular parameters are volume of distribution, blood flow, transit time including mean transit time, and any combination thereof. Volume of distribution is the volume of tissue in the portion of the body in which heat is distributed. Blood flow is the volume of blood moving through the portion of the body per unit time. Transit time is the time required for an individual fluid molecule to flow through the volume of distribution from an arterial input to a venous output. Mean transit time is a bulk property of the fluid and is the average time required for individual fluid molecules to flow through a given region of the part of the body from an arterial input to a venous output. Methods of the present invention include determining a single cardiovascular parameter or multiple cardiovascular parameters.

The cardiovascular parameter can be for a portion of the body, such as an organ or tissue. Non-limiting examples of organs for which a cardiovascular parameter can be determined include the brain, lungs, heart, kidney, liver, stomach and other gastrointestinal organs, and vasculature. Vasculature includes arteries and veins including central and peripheral arteries and veins. For example, the artery can be the carotid artery and the vein can be an internal jugular vein or a large vein draining an organ.

Referring again to FIG. 1, with respect to introducing a fluid into a blood vessel of a patient (10), the fluid can be any biologically compatible fluid that can perfuse the portion of the body. For example, the fluid may be water, blood or a saline solution. The fluid can be introduced over any time frame at any rate sufficient to induce temperature changes that can be effectively imaged. For example, the fluid may be introduced at a constant rate over a period of seconds, such as, for example, a bolus injection where the shape of the input is a square wave. Alternatively, the fluid may be introduced over a period of minutes, where the shape of the input is a desired function of time including a sinusoidal function. Furthermore, the shape of the input may be designed to optimize the arterial input function of the blood vessel being imaged and thereby simplify calculations.

The fluid can be introduced in any manner such that the fluid can perfuse the portion of the body and induce temperature changes that can be effectively imaged. For example, the fluid can be injected intravenously or intra-arterially or introduced as a gas into the lungs via inhalation. Further, the fluid can be introduced at a site local or distant to the portion of the body in which the cardiovascular parameter is being determined. For example, the fluid may be injected into a peripheral vein using a conventional intravenous line, into a central vein using a central venous line, or through a catheter or needle in a peripheral or central artery that supplies the portion of the body in which perfusion is to be determined. The temperature of the introduced fluid can be above or below body temperature. Further, the temperature of the introduced fluid may have a uniform constant temperature below or above body temperature or can vary over time and include temperatures above and below body temperature. For example, the introduced fluid may vary over time when the injection site is remote from the tissue of interest, such as a peripheral vein, and the profile of the injected fluid changes after passing through the heart and pulmonary circulation. Using an injection with a time-varying temperature may reduce such changes. A constant temperature injection may be used, for example, when the injection site is closer to the tissue of interest, such as a central artery, and the profile of the injected fluid does not change as readily.

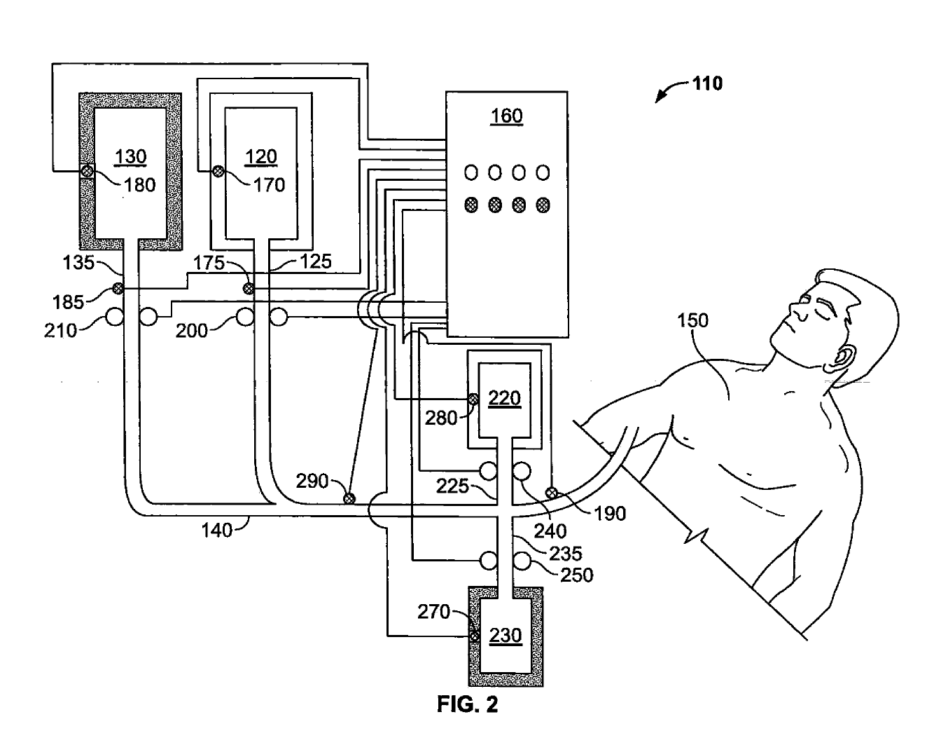

A system can be used for controlling the temperature of the fluid that is introduced into the patient by combining fluids having two different temperatures and introducing the combined fluid into the patient. Referring to FIG. 2, in an embodiment, such a system 110 includes first reservoir 120 containing a first fluid at a temperature below body temperature and second reservoir 130 containing a second fluid at a temperature above body temperature. First and second reservoirs 120 and 130 are in fluid communication with respective first and second fluid lines 125 and 135, which, in turn, are in fluid communication with a convergent line 140. First and second lines 125 and 135 can converge with convergent line 140 via a Y-connector, for example, such that the fluid outflow of reservoirs 120 and 130 is combined into a single fluid line. System 110 further comprises third reservoir 220 containing a third fluid at a temperature below body temperature and fourth reservoir 230 containing a fourth fluid at a temperature above body temperature. Third and fourth reservoirs 220 and 230 are in fluid communication with respective third and fourth fluid lines 225 and 235, which, in turn, are in fluid communication with convergent line 140. Convergent line 140 is insertable into a blood vessel of a patient 150 either directly or indirectly, via a catheter attached to the distal end of convergent line 140.

System 110 further comprises first reservoir temperature sensor 170 in communication with first reservoir 120 and first line temperature sensor 175 in communication with first fluid line 125. System 110 further comprises second reservoir temperature sensor 180 in communication with second reservoir 130 and second line temperature sensor 185 in communication with second fluid line 135. System 110 further comprises third reservoir temperature sensor 280 in communication with third reservoir 220 and fourth reservoir temperature sensor 270 in communication with fourth reservoir 230. In addition, system 110 comprises convergent line temperature sensors 190 and 290. System 110 further comprises controller 160 for controlling the flow of first, second, third and fourth fluids from respective first, second, third and fourth reservoirs 120, 130, 220, and 230. Specifically, in an embodiment, controller 160 is in communication with sensors 170, 180, 175, 185, 190, 270, 280 and 290. Controller 160 is also in communication with first pump 200, second pump 210, third pump 240 and fourth pump 250 which, in turn, are in communication with first fluid line 125, second fluid line 135, third fluid line 225 and fourth fluid line 235 respectively. A non-limiting example of first, second, third and for pumps 200, 210, 240 and 250 are power injectors. In certain embodiments, an exemplary system does not include third and fourth pumps 240 and 250. In order to control the flow of first and second fluids, controller 160 receives temperature input signals from sensors 170, 180, 175, and 185 regarding the temperature of the first and second fluids and accordingly sends out a control signal to pumps 200 and 210 to adjust the flow rate of the fluids. Likewise, in order to control the flow of third and fourth fluids, controller 160 receives temperature input signals from sensors 280 and 270 regarding the temperature of the third and fourth fluids and accordingly sends out a control signal to pumps 240 and 250 to adjust the flow rate of the fluids. Controller 160 may be computerized and the flow rate of first and second fluids exiting respective first and second reservoirs 120 and 130 can be varied in accordance with a look-up table or an algorithm to achieve a desired temperature variation of the introduced combined fluid. Temperature readings from the convergent line temperature sensors 190 and 290 can be used to confirm the expected temperature in convergent line 140 as determined from the look-up table or the algorithm. Controller 160 may be computerized and may introduce additional fluid from third and fourth reservoirs 220 and 230 in accordance with a look-up table or an algorithm to make adjustments to achieve the desired temperature variation of the introduced fluid or to optimize or adjust the leading and trailing edges of the introduced fluid. In one variation of the algorithm used to achieve a desired temperature variation of the fluid, repetitive injections of the fluid can be made and the algorithm adjusted accordingly.

Referring back to FIG. 1, an embodiment of a method of the present invention includes obtaining magnetic resonance information from the portion of the body (20). The magnetic resonance information is determined by physical properties of the portion of the body and includes but is not limited to MR signal intensity, phase information, frequency information and any combination thereof. To obtain such magnetic resonance information, the patient is placed in a NR scanner and radiofrequency (RF) pulses are transmitted to the patient. The RF pulse sequences can be used to excite a slice, a series of slices or a volume of a part of the body. If pulses can be applied in a dynamic fashion so that magnetic resonance information is measured dynamically, such as at multiple sequential points in time. For example, magnetic resonance information can be measured before, during and after the introduced fluid perfuses the portion of the body of the patient. The pulse sequences may include but are not limited to echo-planar, gradient echo, spoiled gradient echo and spin echo. For each slice, series of slices or volume, the magnetic resonance information can be spatially encoded by using magnetic field gradients including phase-encoding gradients and frequency-encoding gradients. Specifically, spatial encoding of the magnetic resonance information can be achieved by applying additional magnetic field gradients after excitation of tissue but before measurement of the magnetic resonance information (phase-encoding gradient) as well as during signal measurement (frequency-encoding gradient). In order to fully spatially encode a slice or volume of excited tissue, the excitation and measurement process can be repeated multiple times with different phase-encoding gradients. When performing a volume acquisition, two different phase encoding gradients can be applied in order to ultimately divide the volume into multiple slices. Spatial encoding allows calculation of the amount of magnetic resonance information emitted by small volume elements (voxels) in the excited slice or volume and therefore allows magnetic resonance information to be measured on a voxel-by-voxel basis in each slice, series of slices or volume.

The magnetic resonance information obtained in 20 is used to determine a magnetic resonance parameter in the portion of the body (30) according to an embodiment of a method of the present invention. The magnetic resonance parameter is determined by the physical properties of the portion of the body and non-limiting examples of magnetic resonance parameters includes phase changes resulting from changes in water proton resonance frequency; changes in T1 relaxation time; changes in diffusion coefficients; phase changes as determined by analysis of spectroscopic data; and any combination thereof. Methods for calculating such magnetic resonance parameters involve using well-known mathematical formulas based on the pulse sequence used and the specific parameter that is to be calculated. Methods of the present invention include measuring a single magnetic resonance parameter or multiple magnetic resonance parameters. The magnetic resonance parameter can be calculated on a voxel-by-voxel basis for each slice, series of slices or volume.

The magnetic resonance parameter calculated in 30 is used to calculate a temperature differential in the portion of the body (40) according to an embodiment of a method of the present invention. Methods for calculating a temperature differential based on the above-identified magnetic resonance parameters are well-known in the art. For example, if the magnetic resonance parameter is phase changes (ΔΦ) corresponding to changes in water proton resonance frequency, a corresponding temperature differential (ΔT) can be calculated in accordance with the equation ΔT=ΔΦ(T)/αΓTEB₀, where a is a temperature dependent water chemical shift in parts per million (ppm) per C⁰, γ is the gyromagnetic ratio of hydrogen, TE is the echo time and B₀ is the strength of the main magnetic field. The temperature differential (ΔT) in a volume of tissue (V) corresponds to a quantity of heat (ΔH) according to the formula ΔH=(ΔT)×(V)×(specific heat)×(specific gravity). The quantity of heat flowing through the arterial input of the part of the body can be calculated by obtaining slices through the arterial input and integrating ΔH over time.

With respect to calculating a temperature differential based on changes in T1 relaxation time, changes in diffusion coefficients, or phase changes as determined by analysis of spectroscopic data such calculations can be performed, for example, in accordance with the methods described by Quesson and Kuroda (e.g. B Quesson, JA de Zwart & CTW Moonen. “Magnetic Resonance Temperature Imaging for Guidance of Thermotherapy;” 12 J Mag Res Img 525 (2000); K Kuroda, RV Mulkern, K Oshio et al. “Temperature Mapping using the Water Proton Chemical Shift; Self-referenced Method with Echo-planar Spectroscopic Imaging;” 43 Magn Reson Med 220 (2000)), both of which are incorporated by reference herein. Of course, as one skilled in the art will appreciate, other methods could also be employed. Notwithstanding which magnetic resonance parameter is used to calculate a temperature differential, the measured temperature change in a voxel will correspond to the concentration of indicator (for example, heat or cold) within the voxel over time.

The temperature differential determined in 40 is used to determine a cardiovascular parameter (50) according to an embodiment of a method of the present invention. Specifically, a temperature differential can be calculated as a function of time, ΔT(t), during a dynamic acquisition. The temperature differential in a voxel of volume V corresponds to a quantity of heat, H(t), according to the formula H(t)=(ΔT(t))×(V)×(specific heat)×(specific gravity). Therefore, a cardiovascular parameter such as quantitative blood flow, F, to an individual voxel can then be determined, for example, according to the formula: (F/V)=H(t)/[AIF(t) [symbol] R(t)], where AIF(t), the arterial input function, is the quantity of heat per unit volume as a function of time at the arterial input to the voxel, R(t) is the residue function and is equal to the fraction of indicator remaining in the voxel at time t, and [symbol] denotes convolution, Such an equation can be solved using a deconvolution technique as described, for example, in L Ostergaard, R M Weisskoff, D A Chesler, C Gyldensted & B R Rosen. “High Resolution Measurement of Cerebral Blood Flow using Intravascular Tracer Bolus Passages. Part I: Mathematical Approach and Statistical Analysis.” 36 Magn Res Med 715 (1996), which is incorporated by reference herein. Alternatively, an exponential approximation can be used to calculate quantitative flow, F, for example, where the descending portion of H(t) is an exponential function such that H(t)=H₀ exp(−kt), where H₀ is the quantity of heat at time t=0 and k is a constant. By definition, k=F/V and k is then calculated based on the observed decay of H(t).

A cardiovascular parameter, such as qualitative blood flow, F, to an individual voxel can be measured, for example, according to the formula:

$$F ∝ 1 / {\int }_{0}^{∞} H ( t ) d t .$$

A cardiovascular parameter, such as mean transit time, MTT, corresponding to an individual voxel can be determined, for example, according to the formula: MTT=V/F. A cardiovascular parameter, such as volume of distribution, V, of an individual voxel can be measured, for example, according to the formula: V=(slice thickness)×(field of view)²/[(phase matrix size)×(frequency matrix size)]. Of course, other methods for determining a cardiovascular parameter will be known to one of skill in the art and the above-mentioned methods are only exemplary.

In an embodiment of a method of the present invention, a determined cardiovascular parameter can be used to produce an image in which a brightness or a color of pixels therein is determined by the cardiovascular parameter. Such an image can be produced by display systems by following methods well-known in the art, such as the method described by C Warmuth, M Gunther & C Zimmer; “Quantification of Blood Flow in Brain Tumors: Comparison of Arterial Spin Labeling and Dynamic Susceptibility weighted Contrast-enhanced MR Imaging;” 228 Radiology 523 (2003), for example, which is incorporated by reference herein. For example, pixel brightness can be set equal to a linear multiple of the quantitative or the qualitative blood flow. Alternatively, pixel color can be varied to indicate higher values of flow in red and lower values of blood flow in blue on a sliding color scale.

In another embodiment, the present invention provides a machine-readable medium having stored thereon a plurality of executable instructions, which, when executed by a processor, performs obtaining magnetic resonance information from a portion of a body of a patient after introduction of fluid into a blood vessel of the patient. The plurality of executable instructions further performs determining a magnetic resonance parameter in the portion of the body using the magnetic resonance information, determining a temperature differential in the portion of the body using the magnetic resonance parameter, and determining a cardiovascular parameter using the temperature differential.

Referring to FIG. 3, the above mentioned method may be performed by a user computing device 300 such as a MRI machine, workstation, personal computer, handheld personal digital assistant (“PDA”), or any other type of microprocessor-based device. User computing device 300 may include a processor 310, input device 320, output device 330, storage device 340, client software 350, and communication device 360. Input device 320 may include a keyboard, mouse, pen-operated touch screen, voice-recognition device, or any other device that accepts input. Output device 330 may include a monitor, printer, disk drive, speakers, or any other device that provides output. Storage device 340 may include volatile and nonvolatile data storage, including one or more electrical, magnetic or optical memories such as a RAM, cache, hard drive, CD-ROM drive, tape drive or removable storage disk. Communication device 360 may include a modem, network interface card, or any other device capable of transmitting and receiving signals over a network. The components of user computing device 300 may be connected via an electrical bus or wirelessly. Client software 350 may be stored in storage device 340 and executed by processor 310, and may include, for example, imaging and analysis software that embodies the functionality of the present invention.

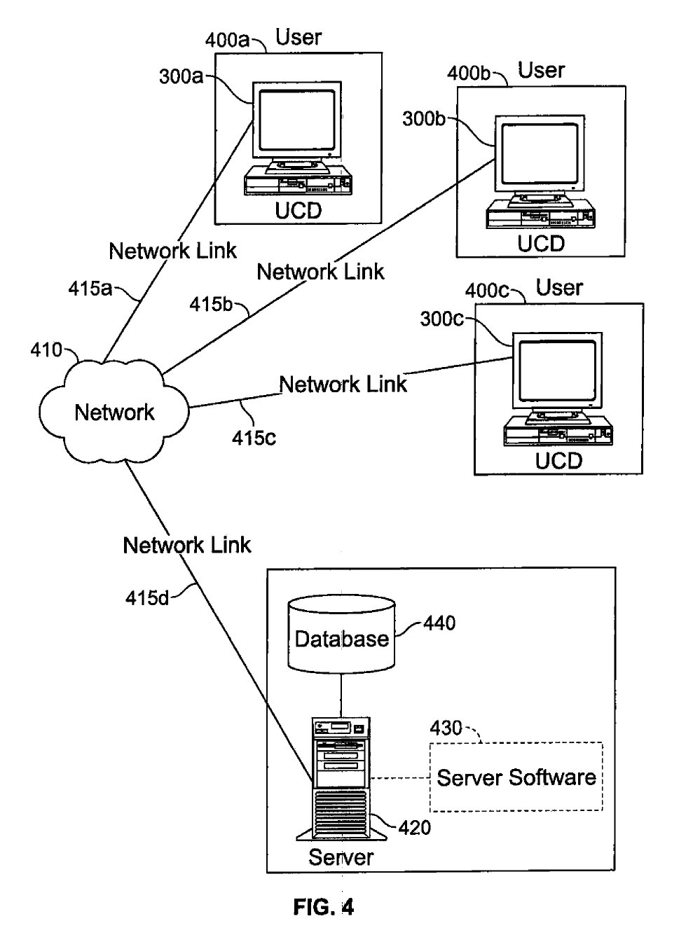

Referring to FIG. 4, the analysis functionality may be implemented on more than one user computing device 300 via a network architecture. For example, user computing device 300 may be an MRI machine that performs all determination, calculation and measurement functionality. In another embodiment, user computing device 300a may be a MRI machine that performs the magnetic resonance information measurement functionality and the magnetic resonance parameter determination functionality, and then transfers this determination over network 410 to server 420 or user computing device 300b or 300c for determination of a temperature differential and cardiovascular parameter, for example. The determined cardiovascular parameter could further be transferred to another user computing device 300 belonging to the patient or another medical services provider for further analysis.

Referring again to FIG. 4, network link 415 may include telephone lines, DSL, cable networks, T1 or T3 lines, wireless network connections, or any other arrangement that implements the transmission and reception of network signals. Network 410 may include any type of interconnected communication system, and may implement any communications protocol, which may secured by any security protocol. Server 420 includes a processor and memory for executing program instructions, as well as a network interface, and may include a collection of servers. Server 420 may include a combination of servers such as an application server and a database server. Database 440 may represent a relational or object database, and may be accessed via server 420.

User computing device 300 and server 420 may implement any operating system, such as Windows or UNIX. Client software 350 and server software 430 may be written in any programming language, such as ABAP, C, C++, Java or Visual Basic.

### Example 1

An MRI model was used to simulate flow through a capillary bed. The model included a cellulose triacetate hollow fiber dialyzer that was continuously perfused with saline at room temperature. A portion of the dialysis tubing simulated a tissue capillary bed and the continuous perfusion simulated blood flow through the cardiovascular system of the body. The model also contained a port that allowed injection of a fluid bolus into the dialysate. A power injector was utilized to inject the fluid bolus. The portion of the dialysis tubing simulating the tissue capillary bed was placed in a 1.5 T MR scanner. MR-compatible thermometers were placed proximal (thermometer 1) and distal (thermometer 2) to the simulated capillary bed with respect to the direction of flow such that fluid flowed past thermometer 1 before it flowed past thermometer 2. The port that allowed injection of the fluid bolus was placed proximal to thermometer 1 with respect to the direction of flow. A dynamic gradient echo scan was utilized to monitor the passage of the fluid bolus.

Three power-injected boluses of 30 ml of ice cold saline (4° C.) and three power-injected boluses of 60 ml of room temperature saline were administered. Prior to each injection of a fluid bolus, a baseline set of MR phase images were obtained through the simulated capillary bed and these images were used as reference image for calculation of phase changes. Additional phase images were obtained for each fluid bolus injection. The phase images were constructed on a voxel-by-voxel basis. For each fluid bolus, a temperature difference was calculated between the dynamic phase images and the reference image on a voxel-by-voxel basis using the formula ΔT=ΔΦ(T)/αγTEB₀, where ΔΦ(T) is the calculated phase change, α is a temperature dependent water chemical shift in ppm per C⁰, γ is the gyromagnetic ratio of hydrogen, TE is the echo time and B₀ is the strength of the main magnetic field.

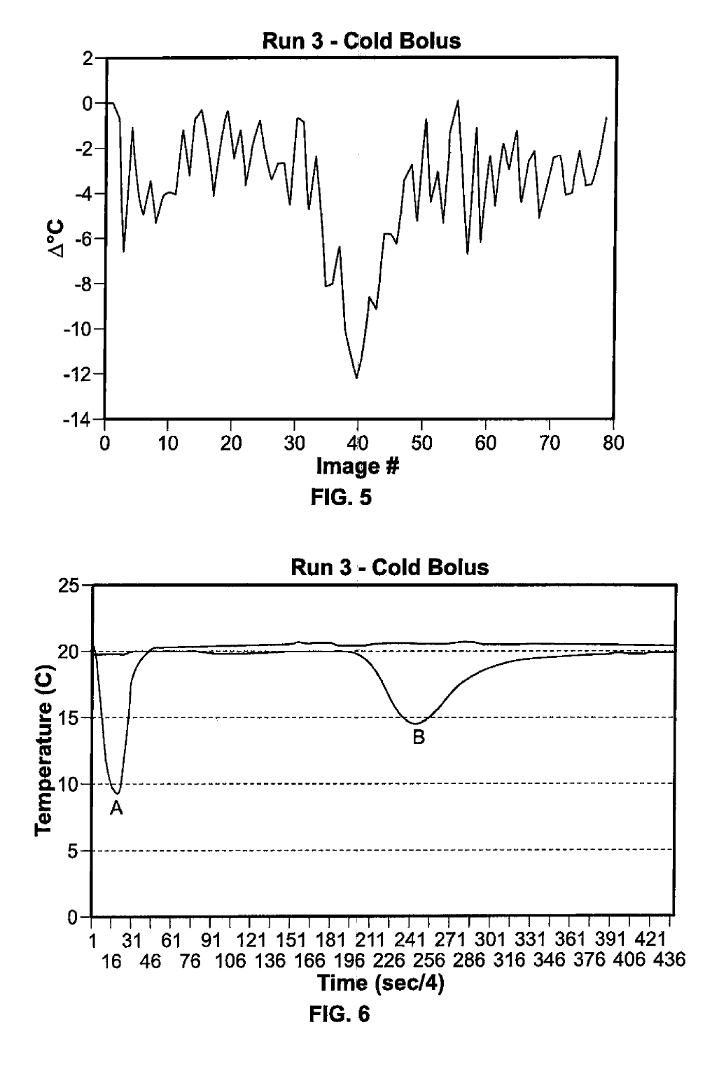

FIG. 5 is a graph of the calculated temperature differentials in sequential dynamic phase images as a function of time following an injection of a cold saline bolus. The well-defined trough in the curve corresponds to the lowest calculated temperature differential following a cold saline bolus. FIG. 6 is a graph showing the measured temperature as a function of time at thermometer 1 (A) and thermometer 2 (B) that corresponds to the cold saline bolus of FIG. 5 as the fluid bolus of cold saline passes by thermometers 1 and 2. Curve A (with the deeper trough and more narrow range) corresponds to the temperature changes over time as the fluid bolus of cold saline passes by thermometer 1 (proximal to the simulated capillary bed). Curve B (with the shallower trough and wider range) corresponds to the temperature changes over time as the fluid bolus of cold saline passes by thermometer 2 (distal to the simulated capillary bed).

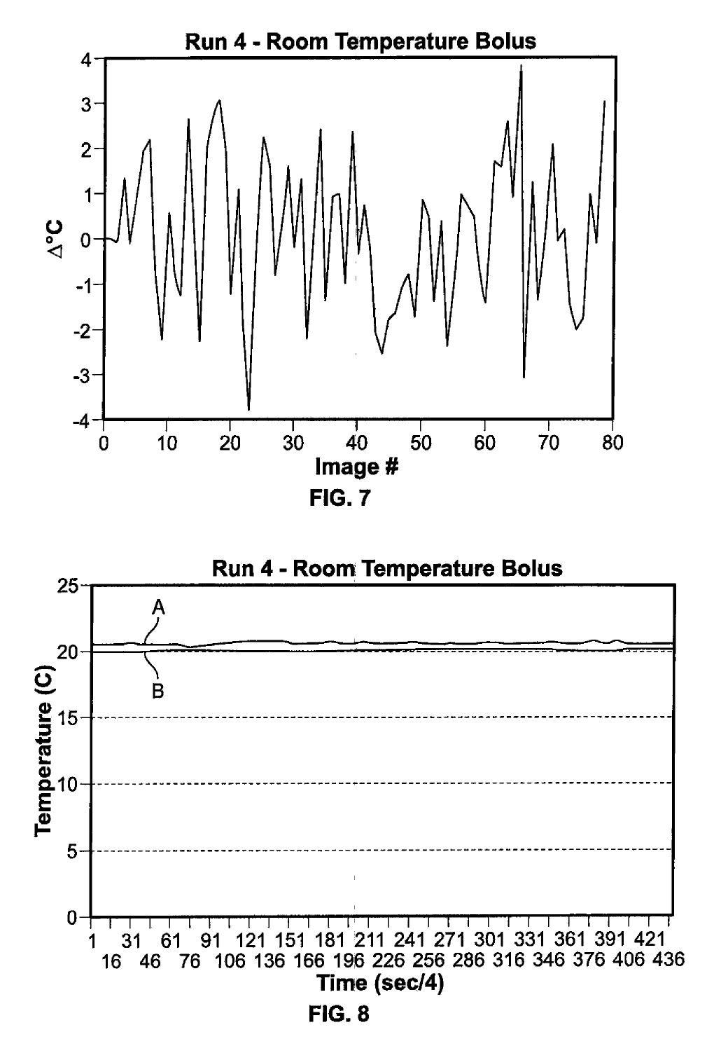

FIG. 7 is a graph of calculated temperature changes in sequential dynamic phase images as a function of time following an injection of a room temperature saline bolus. The random high frequency and low amplitude changes in the curve correspond to random fluctuations in temperature measurements due to noise. FIG. 8 is a graph showing the measured temperature as a function of time at thermometer 1 (A) and thermometer 2 (B) that corresponds to the room temperature bolus of FIG. 7. The curve remains essentially flat corresponding to no significant temperature change over time at either thermometer.

Based on this simulation model, temperature sensitive MRI measurements corresponded closely to the temperature changes detected by thermometers when a bolus of cold fluid was injected into a simulated cardiovascular system. For example, the maximal calculated decrease in temperature of FIG. 5 was approximately 12° C. and this corresponds almost exactly to the maximal decrease in temperature of curve A in FIG. 6. Furthermore, temperature sensitive MRI correctly determined that there was no temperature change when a bolus of fluid at the same temperature as the fluid in the simulated cardiovascular system was injected.

The foregoing description and example have been set forth merely to illustrate the invention and are not intended as being limiting. Each of the disclosed aspects and embodiments of the present invention may be considered individually or in combination with other aspects, embodiments, and variations of the invention. In addition, unless otherwise specified, none of the steps of the methods of the present invention are confined to any particular order of performance. Modifications of the disclosed embodiments incorporating the spirit and substance of the invention may occur to persons skilled in the art and such modifications are within the scope of the present invention. Furthermore, all references cited herein are incorporated by reference in their entirety.

What is claimed is:

1. A method for determining a cardiovascular parameter of a portion of a body of a patient comprising;
introducing a fluid into a blood vessel of the patient;
obtaining magnetic resonance information from the portion of the body;
determining a magnetic resonance parameter from the portion of the body using the magnetic resonance information;
determining a temperature differential in the portion of the body using the magnetic resonance parameter; and
determining the cardiovascular parameter using the temperature differential.

2. The method of claim 1, wherein the cardiovascular parameter is blood flow.

3. The method of claim 2, wherein the blood flow is cerebral blood flow.

4. The method of claim 1, wherein the cardiovascular parameter is volume of distribution.

5. The method of claim 4, wherein the blood volume is cerebral volume of distribution.

6. The method of claim 1, wherein the cardiovascular parameter is transit time.

7. The method of claim 6, wherein the cardiovascular parameter is mean transit time.

8. The method of claim 1, wherein the portion of the body is an organ.

9. The method of claim 8, wherein the organ is a brain.

10. The method of claim 1, wherein obtaining the magnetic resonance information comprises:
placing the patient in a magnetic resonance scanner;
transmitting radiofrequency pulses to the patient to excite a slice, a series of slices or a volume containing the portion of the body; and
measuring the magnetic resonance information from the portion of the body.

11. The method of claim 1, wherein introducing the fluid comprises introducing the fluid at a temperature below body temperature of the patient.

12. The method of claim 1, wherein introducing the fluid comprises introducing the fluid at a temperature that varies over time.

13. The method of claim 12, wherein the temperature that varies over time includes any combination of a temperature above, a temperature below and a temperature equal to body temperature of the patient.

14. The method of claim 1, wherein obtaining the magnetic resonance information comprises collecting the magnetic resonance information at multiple sequential points in time from the portion of the body after introducing the fluid.

15. The method of claim 14, wherein collecting the magnetic resonance information at multiple sequential points comprises collecting the magnetic resonance information before, during and after the introduced fluid perfuses the portion of the body of the patient.

16. The method of claim 1, wherein obtaining the magnetic resonance information comprises obtaining the magnetic resonance information on a slice-by-slice or volume basis through the portion of the body of the patient.

17. The method of claim 1, wherein determining the magnetic resonance parameter comprises determining the magnetic resonance parameter on a voxel-by-voxel basis through the portion of the body of the patient.

18. The method of claim 1, wherein the magnetic resonance parameter comprises changes in water proton resonance frequency and the temperature differential is determined using the changes in water proton resonance frequency.

19. The method of claim 1, wherein the magnetic resonance parameter comprises changes in T1 relaxation time of water protons and the temperature differential is determined using the changes in T1 relaxation time.

20. The method of claim 1, wherein the magnetic resonance parameter comprises changes in a diffusion coefficient of water in the portion of the body and the temperature differential is determined using the changes in the diffusion coefficient.

22. The method of claim 1, wherein the magnetic resonance parameter comprises changes in magnetic resonance spectroscopy measurements of the portion of the body and the temperature differential is determined using the changes in magnetic resonance spectroscopy measurements.

23. The method of claim 1 further comprising:
producing an image in which a brightness or a color of pixels therein is determined by the cardiovascular parameter.

24. A method for determining a cardiovascular parameter of a portion of a body of a patient comprising:
introducing a gas into a lung of the patient;
obtaining magnetic resonance information from the portion of the body;
determining a magnetic resonance parameter from the portion of the body using the magnetic resonance information;
determining a temperature differential in the portion of the body using the magnetic resonance parameter; and
determining the cardiovascular parameter using the temperature differential

25. A machine-readable medium having stored thereon a plurality of executable instructions, which, when executed by a processor, perform the following:
obtaining magnetic resonance information from a portion of a body of a patient after introduction of fluid into a blood vessel of the patient;
determining a magnetic resonance parameter from the portion of the body using the magnetic resonance information;
determining a temperature differential in the portion of the body using the magnetic resonance parameter; and
determining a cardiovascular parameter using the temperature differential.

26. The machine-readable medium of claim 25, wherein determining a magnetic resonance parameter in the portion of the body comprises measuring the magnetic resonance information on a voxel-by-voxel basis.

27. The machine-readable medium of claim 25, wherein obtaining the magnetic resonance information comprises obtaining the magnetic resonance information before, during and after blood perfuses the portion of the body.

28. The machine-readable medium of claim 25, wherein the magnetic resonance parameter comprises changes in water proton resonance frequency and the temperature differential is determined using the changes in water proton resonance frequency.

29. A system for determining a cardiovascular parameter of a portion of a body of a patient comprising:
means for introducing a fluid into a blood vessel of the patient;
means for obtaining magnetic resonance information from the portion of the body;
means for determining a magnetic resonance parameter from the portion of the body using the magnetic resonance information;
means for determining a temperature differential in the portion of the body using the magnetic resonance parameter; and
means for determining the cardiovascular parameter using the temperature differential.

30. The system of claim 29, wherein the means for introducing a fluid comprises a central arterial catheter.

31. The system of claim 29, wherein the means for introducing a fluid comprises a central venous catheter.

32. The system of claim 29, wherein the means for introducing a fluid comprises a peripheral venous catheter.

33. The system of claim 29, wherein the means for determining a temperature differential comprises means for calculating changes in water proton resonance frequency using the changes in water proton resonance frequency to determine the temperature differential.
